(19) United States
(12) Patent Application Publication
(10) Pub. No.: US 2022/0118150 A1
(43) Pub. Date: Apr. 21, 2022

(54) MULTI-FUNCTIONAL PLASMA DRIVEN CATALYST SYSTEM

(71) Applicant: Nano and Advanced Materials Institute Limited, Hong Kong (CN)

(72) Inventors: Lok Hang KEUNG, Hong Kong (CN); Juanfang CAI, Hong Kong (CN); King Ho SO, Hong Kong (CN); Ka Kit YEE, Hong Kong (CN); Ka Chun LEE, Hong Kong (CN)

(21) Appl. No.: 17/451,164

(22) Filed: Oct. 18, 2021

Related U.S. Application Data
(60) Provisional application No. 63/198,467, filed on Oct. 21, 2020.

(51) Int. Cl. A61L 9/22; A61L 2/14; H05H 1/24

(52) U.S. Cl. CPC A61L 9/22; A61L 2/14; B01D 53/32; H05H 2245/15; H05H 1/2406

(57) ABSTRACT

A plasma driven catalyst apparatus useful for disinfecting and purifying air. The apparatus has a synergistically favorable effect from plasma and catalyst on high disinfecting and purifying efficiency and efficacy, low by-product formation, and low energy consumption. The plasma combined with catalyst enhances the production of new reactive species, increases the oxidizing power of the plasma discharge, as well as activate the catalyst that additionally contributes towards the disinfection and purification process and the elimination of toxic by-products.

### CROSS-REFERENCE TO RELATED APPLICATIONS

The present application claims priority to U.S. provisional patent application Ser. No. 63/198,467, filed on Oct. 21, 2020, and the disclosure of which is incorporated herein by reference in its entirety.

### TECHNICAL FIELD

The present disclosure generally relates to a plasma driven catalyst (PDC) system useful for air purification and an apparatus comprising the same.

### BACKGROUND

Clean air is a basic requirement of life. The quality of air inside homes, offices, schools, public buildings, health care facilities or other private and public buildings where people spend a large part of their life is an essential determinant of healthy life and people's well-being. Exposure to hazardous substances, such as NOₓ, SOₓ, odor, volatile organic compounds (VOCs), bacteria and viruses, can lead to a broad range of health problems and may even be fatal.

Non-thermal plasma (NTP) technology is currently being investigated for the removal of SO₂, NOₓ, odors, and VOCs. However, NTP alone has many drawbacks, such as low energy efficiency, poor selectivity with carbon dioxide, and byproduct formation. PDCs provide a synergistic combination of NTP with a catalyst can overcome these limitation. The catalyst reacts with incoming air/gas and moisture to generate reactive oxygen species (ROS), such as peroxide, hydroxyl, and superoxide radicals to dissociate pollutants. These ROS are highly effective in killing bacteria, virus and mold, as well as act as a repellent. Plasma systems can rapidly dissociate air pollutants under ambient temperature and pressure, but typically cannot handle large volume of air, because a conventional air purification apparatus would induce high pressure drop in a fluid.

The main disadvantages of the plasma-based processes typically include the emission of unwanted byproducts and low energy yield of the systems. Attempts have been made to overcome these problems by combining non-thermal plasma with catalysis. Many efforts have been devoted to optimize the plasma-catalysis synergy. Additionally, the function and efficiency of PDC reactors are also effected by various elements ranging from catalyst and its supported material to system electrical and structure design, which can further complicate the development of efficient and effective PDC reactors.

There thus exists a need for improved PDC reactors that address or overcome at least some of the aforementioned challenges.

### SUMMARY

The present disclosure relates to a multi-functional PDC reactor useful for air treatment and removal of VOC, odor, disinfection, mold, bacteria, viruses and pests from the air. The PDC reactors described herein can be used in air purifiers or in HVAC systems for, e.g., residential homes, hospitals, office buildings and shopping malls.

In certain embodiments, the PDC reactor comprises a mesh-plate type PDC reactor integrated with titanium dioxide (TiO₂) based optionally doped catalyst supported by metal or ceramic foam.

In a first aspect provided herein is a plasma driven catalyst (PDC) reactor comprising: at least two spaced air permeable plasma electrodes for generating plasma within a plasma zone between the at least two spaced air permeable plasma electrodes by an alternating current voltage; at least one substrate supported catalyst comprising a foam substrate selected from a metal foam and a ceramic foam; and at least one TiO₂ photocatalyst layer coated on at least one surface of the foam substrate; and at least one air inlet and at least one air outlet for allowing air to pass through the at least two spaced air permeable plasma electrodes and the plasma zone, wherein the one substrate supported catalyst is disposed within the plasma zone and on a surface of at least one of the at least two spaced air permeable plasma electrodes.

In a first embodiment of the first aspect, provided herein is the PDC reactor of the first aspect, wherein the metal foam is selected from stainless steel, aluminum, nickel, copper, gold, and their alloys; and the ceramic foam is selected from silicon carbide, boron carbide, hafnium carbide, tantalum carbide, zirconia, alumina, hafnium dioxide, magnesium oxide, silicon dioxide, yttria, silicon nitride, aluminum nitride, boron nitride, hafnium nitride, titanium boride, cordierite, mullite, and mixtures thereof.

In a second embodiment of the first aspect, provided herein is the PDC reactor of the first aspect, wherein the foam substrate has a porosity between than 70% to 95%.

In a third embodiment of the first aspect, provided herein is the PDC reactor of the first aspect, wherein the foam substrate has an average pore size between 450 to 3,000 μm.

In a fourth embodiment of the first aspect, provided herein is the PDC reactor of the first aspect, wherein the foam substrate has a geometric surface area between 5,000 to 15,000 m²/m³.

In a fifth embodiment of the first aspect, provided herein is the PDC reactor of the first aspect, wherein the at least one TiO₂ photocatalyst layer further comprises a dopant selected from the group consisting of titanium, zirconium, copper, manganese, lanthanum, molybdenum, tungsten, vanadium, selenium, barium, cesium, tin, iron, magnesium, gold, cobalt, nickel, palladium, their oxides thereof, or their alloys thereof.

In a sixth embodiment of the first aspect, provided herein the PDC reactor of the first aspect, wherein the at least one TiO₂ photocatalyst layer further comprises a dopant selected from nickel(II)oxide, copper(II)oxide, and cobalt(II)oxide.

In a seventh embodiment of the first aspect, provided herein is the PDC reactor of the sixth embodiment of the first aspect, wherein the dopant is present in the at least one TiO₂ photocatalyst layer between 4% to 40% by weight.

In an eighth embodiment of the first aspect, provided herein is the PDC reactor of the first aspect, wherein the at least one TiO₂ photocatalyst layer is prepared by a sol-gel deposition method.

In a ninth embodiment of the first aspect, provided herein is the PDC reactor of the first aspect, wherein the at least two spaced air permeable plasma electrodes are mesh-plate electrodes.

In a tenth embodiment of the first aspect, provided herein is the PDC reactor of the ninth embodiment of the first aspect, wherein each of the at least two spaced air permeable plasma electrodes are spaced between 1 mm to 50 mm from each other.

In an eleventh embodiment of the first aspect, provided herein is the PDC reactor of the first aspect, wherein the foam substrate is a stainless steel foam having a porosity between 75% to 90%; the at least one TiO₂ photocatalyst layer further comprises 4% to 40% by weight of a dopant selected from nickel(II)oxide, copper(II)oxide, and cobalt(II)oxide; and the at least two spaced air permeable plasma electrodes are mesh-plate electrodes spaced from each other at a distance between 8 to 12 mm.

In a twelfth embodiment of the first aspect, provided herein is the PDC reactor of the first aspect, wherein the foam substrate is a nickel alloy foam having a porosity between 75% to 90%; and the at least two spaced air permeable plasma electrodes are mesh-plate electrodes spaced from each other at a distance between 8 to 12 mm.

In a thirteenth embodiment of the first aspect, provided herein is the PDC reactor of the first aspect further comprising an alternating current power supply connected to the at least two spaced air permeable plasma electrodes, wherein the alternating current power supply provides a frequency ranging from 0.1 to 30 kHz and the alternating voltage ranging from 1 to 10 kV.

In a fourteenth embodiment of the first aspect, provided herein is the PDC reactor of twelfth embodiment of the first aspect, further comprising an alternating current power supply connected to the at least two spaced air permeable plasma electrodes, wherein the alternating current power supply provides a frequency ranging from 1 to 10 kHz and the alternating voltage ranging from 1 to 5 kV.

In a second aspect, provided herein is an air purifier comprising the PDC reactor of the first aspect.

In a first embodiment of the second aspect, provided herein is the air purifier of the second aspect further comprising: an electric fan positioned to direct air through the at least one air inlet.

In a third aspect, provided herein is a method of treating air using the PDC reactor of the first aspect, the method comprising: directing air into the at least one air inlet, applying an alternating current voltage to the at least two spaced air permeable plasma electrodes thereby generating plasma in the plasma zone; and allowing the air to pass through the plasma zone and out of the at least one air outlet thereby forming treated air.

In a first embodiment of the third aspect, provided herein is the method of the first aspect, wherein the air comprises or is suspected of comprising one or more contaminants selected from the group consisting of a volatile organic compound, formaldehyde, CO, NO₂, H₂S, NH₃, NO, an odor, and a microorganism.

In a second embodiment of the third aspect, provided herein is the method of the first aspect further comprising contacting seeds or a surface with the treated air, wherein the surface is contaminated or suspected of being contaminated with at least one microorganism.

Other aspects and advantages of the present invention will be apparent to those skilled in the art from a review of the ensuing description.

### BRIEF DESCRIPTION OF THE DRAWINGS

The above and other objects and features of the present invention will become apparent from the following description of the invention, when taken in conjunction with the accompanying drawings, in which:

FIG. 1 depicts a schematic of an exemplary PDC reactor (A) with the TiO₂ coated 106 foam 107 is positioned after the plasma zone 105 according to certain embodiments described herein; and (B) with the TiO₂ coated 106 foam layer 107 is positioned before the plasma zone 105 according to certain embodiments described herein.

FIG. 2 depicts a schematic of an exemplary PDC reactor according to certain embodiments described herein.

FIG. 3 depicts an exemplary reaction flow chart outlining the steps to prepare the TiO₂ precursor solution for applying the TiO₂ coating to the metallic or ceramic foam in accordance with certain embodiments described herein.

FIG. 4 depicts an exemplary reaction flow chart outlining the steps to apply the (A) the TiO₂ coating to the metallic or ceramic foam in accordance with certain embodiments described herein; and (B) the TiO₂ coating further comprising a metal dopant to the metallic or ceramic foam in accordance with certain embodiments described herein.

FIG. 5 depicts scanning electron microscopy images of the surface of the (A) TiO₂ doped with CuO coated ceramic foam, (B) TiO₂ doped with CoO coated ceramic foam, and (C) TiO₂ doped with NiO coated ceramic foam in accordance with certain embodiments described herein.

FIG. 6 depicts a graph showing the real-time wave form, peak to peak voltage and frequency of a PDC reactor in accordance with certain embodiments described herein.

FIG. 7 depicts a table showing testing results of PDC reactors with TiO₂ coated on metal foam on the removal of VOCs, HCHO, CO, NO₂, H₂S, NH₃, and NO in accordance with certain embodiments described herein. TiO₂ coating is incorporated in this PDC reactors and formed on 5 mm thick nickel alloy foam by using sol-gel method which is shown in FIG. 3. This PDC reactor comprises two SS340 mesh plates as electrodes in parallel. The TiO₂ coated alloy foam is embedded on cathode mesh plate. The mesh-to mesh distance was 8 mm. The cross area of the PDC unit is 0.0225 m².

FIG. 8 depicts a table showing testing results of PDC reactors on the removal of viruses in accordance with certain embodiments described herein. The PDC reactors tested in this condition is the same as that in FIG. 7.

FIG. 9 depicts a table showing testing results of PDC reactors including CuO, CoO, and NiO doped TiO₂ analyzed in FIG. 11 on the removal of HCHO in accordance with certain embodiments described herein. For this reactor, CuO, CoO, and NiO doped TiO₂ catalyst is applied on ceramic foam. The doping procedure is illustrated in FIG. 4.

FIG. 10 depicts a table showing testing results of PDC reactors on the removal of HCHO and O₃ emission in accordance with certain embodiments described herein.

FIG. 11 depicts the (top) energy-dispersive X-ray (EDX) spectrum of a CuO doped TiO₂ coated ceramic foam catalyst and (bottom) elemental analysis of the TiO₂ coating based on the EDX data in accordance with certain embodiments described herein.

FIG. 12 depicts the (top) energy-dispersive X-ray (EDX) spectrum of a NiO doped TiO₂ coated ceramic foam catalyst and (bottom) elemental analysis of the TiO₂ coating based on the EDX data in accordance with certain embodiments described herein.

FIG. 13 depicts the (top) energy-dispersive X-ray (EDX) spectrum of a CoO doped TiO₂ coated ceramic foam catalyst and (bottom) elemental analysis of the TiO₂ coating based on the EDX data in accordance with certain embodiments described herein.

FIG. 14 depicts a schematic of an exemplary air purifier comprising a PDC reactor in accordance with certain embodiments described herein.

FIG. 15 depicts a photograph and corresponding schematic of a PDC reactor in accordance with certain embodiments described herein. A photograph of the spaced air permeable plasma electrode is depicted with the dimensions of an exemplary mesh grill type spaced air permeable plasma electrode.

FIG. 16 depicts photographs (top) and a table (bottom) showing testing results of PDC reactors on spinach (A) and kale (B) seed germination improvement in accordance with certain embodiments described herein.

FIG. 17 depicts on-site photo records of PDC reactors efficacy on fly and fruit fly pest repellence in accordance with certain embodiments described herein.

FIG. 18 depicts a table showing testing results of PDC reactors on surface bacteria killing efficiency in accordance with certain embodiments described herein.

### DETAILED DESCRIPTION

Optimization of the configuration in electrode shape, catalyst type and supported foam material for the present disclosure was developed to achieve multi-functional PDC reactor useful for air treatment with less energy consumption. The catalyst used in this PDC can be titanium dioxide (TiO₂) doped with other metal oxides. This TiO₂-based catalyst can be activated in the plasma reactor without additional UV light irradiation. The generated ozone and other by products from the reactor can be eliminated by the TiO₂ based catalyst. The TiO₂ catalyst is supported by metal/ceramic foam which of the sponge-like structure has as very large surface area per unit weight as catalyst carriers. The high porosity of foam material also achieve to a low resistant in fluid flow. The electrodes and foam can be rectangular or square in shape for facilitating stacking and also provide the advantage of ease of fabrication and scaling-up in a HVAC system.

In this disclosure, TiO₂ is selected as a photocatalyst for the PDC reactor. Alternatively, TiO₂ doped with different metal oxides, such as NiO, CoO, and CuO, can also be used to enhance the photocatalytic performance of the PDC reactor. Optionally doped TiO₂ catalysts can be coated on a foam substrate, such as metal foam, glass, ceramic foam, fabric, and mixtures thereof. Metal and ceramic foams can have a cellular structure consisting of a solid metal or ceramic materials respectively with gas-filled pores comprising a large portion of the volume. Because of their high porosity, large specific surface area, and satisfactory thermal and mechanical stability, metal and ceramic foam are considered as effective catalyst carriers PDC reactors described herein.

Apart from selecting an improved catalyst and support material, it is also vital to provide a stable and effective high voltage output for the PDC reactor. To increase the energy density and reduce pressure drop, a metal mesh-plate type electrode is used in certain embodiments of the PDC reactors described herein. The configuration of electrode size, distance and power supply was examined and optimized. Referring to optimal configuration, easy adjustment and scaling-up could be achieved to meet different situations of flow rate, duct size and other engineering specification.

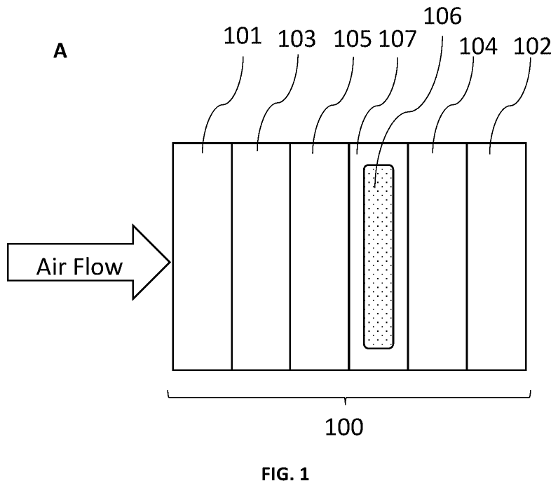

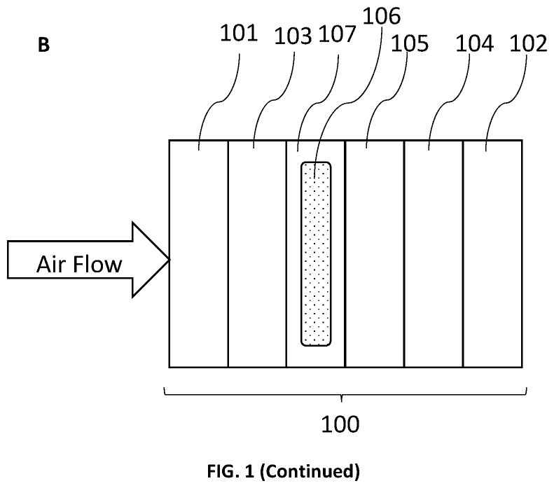

FIGS. 1A and 1B depict exemplary schematics of the PDC reactor 100, which comprises at least two spaced air permeable plasma electrodes 101 and 103 for generating plasma within a plasma zone 105 between the at least two spaced air permeable plasma electrodes by an alternating current voltage; at least one substrate supported catalyst 106, 107 comprising a foam substrate 106 selected from a metal foam and a ceramic foam; and at least one TiO₂ photocatalyst layer 107 coated on at least one surface of the foam substrate; and at least one air inlet 101 and at least one air outlet 102 for allowing air to pass through the at least two spaced air permeable plasma electrodes and the plasma zone, wherein the at least one substrate supported catalyst is disposed within the plasma zone and on a surface of at least one of the at least two spaced air permeable plasma electrodes.

Advantageously, the PDC reactor is configured such that air flows through the at least two spaced air permeable plasma electrodes 103 and 104, plasma zone 105, and around and through the at least one substrate supported catalyst 106, 107 thus providing optimal exposure time of the air flowing through the PDC reactor to the plasma zone 105 and the at least one substrate supported catalyst 106, 107, which can result in an increase in PDC reactor performance.

FIGS. 1A and 1B depict two configurations of an exemplary PDC reactor 100, wherein the location of the at least one substrate supported catalyst 106, 107 is placed after the plasma zone 105 (FIG. 1A) or placed before the plasma zone 105 (FIG. 1). Alternatively the at least one substrate supported catalyst can be positioned at the front end, middle, or back end of the plasma reactor.

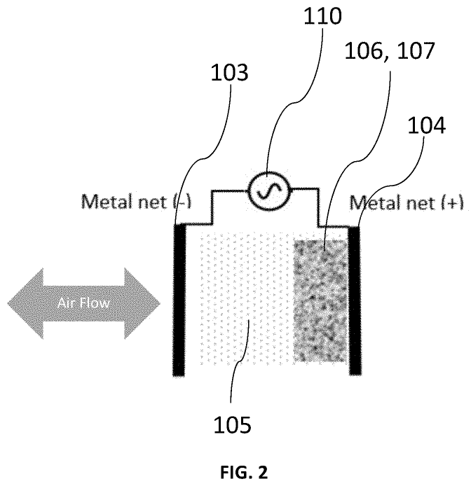

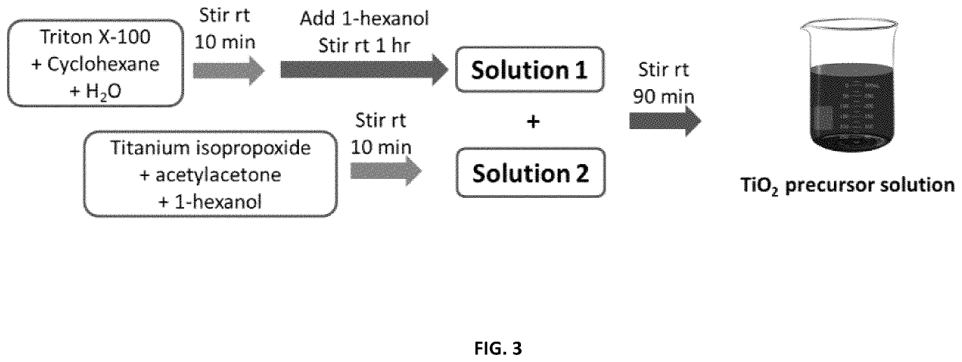

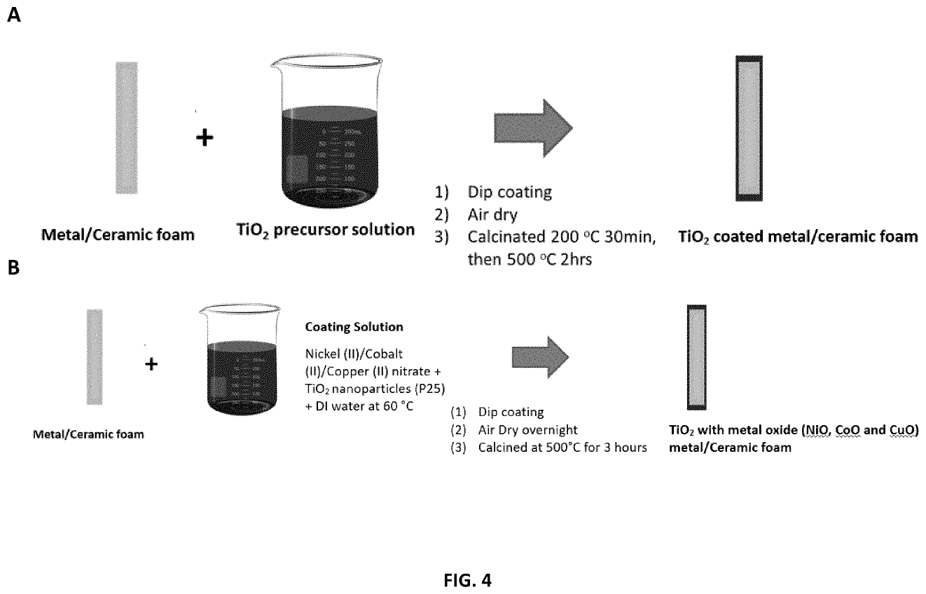

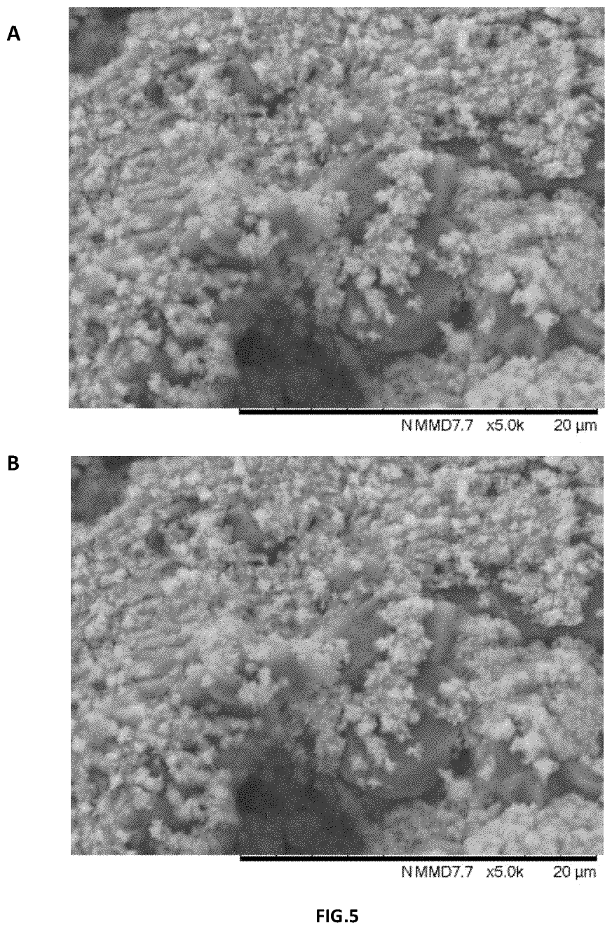

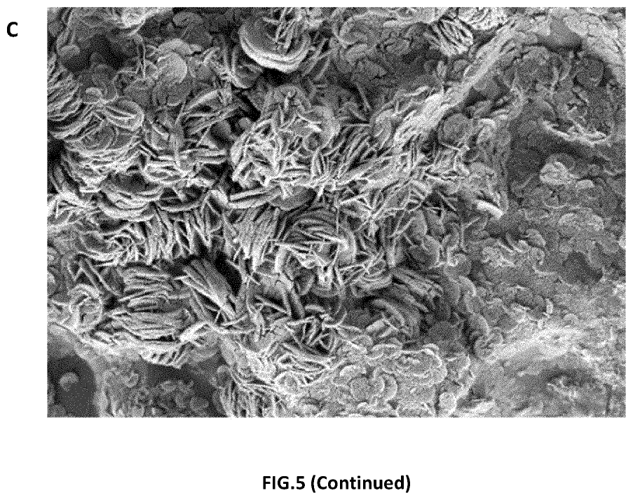

FIG. 2 depicts a schematic of an exemplary PDC reactor comprising at least two spaced air permeable plasma electrodes 103 and 104, plasma zone 105 between the at least two spaced air permeable plasma electrodes by an alternating current voltage 110, and the at least one substrate supported catalyst 106, 107.

The foam substrate can be selected from metal foams, ceramic foams, and mixtures thereof. In certain embodiments, the metal foam is selected from the group consisting of stainless steel, aluminum, nickel, iron, and their alloys. The ceramic foam can be selected from silicon carbide, boron carbide, hafnium carbide, zirconia, aluminum oxide, hafnium dioxide, magnesium oxide, silicon dioxide, and mixtures thereof. In certain embodiments, the foam substrate is selected from stainless steel foam, nickel alloy foam, aluminum oxide foam, and mixtures thereof.

The foam substrate can have an open cell structure, closed cell structure, or a combination thereof. The pores of the foam substrate can be regularly shaped or irregularly shaped, including pores having sphere, plate, cylinder, disc, ring, star and other shapes. The porosity of the foam substrate can range from 75-98%. The average pore size of the foam substrate can range from an average pore size between 450 to 3,000 μm. The foam substrate can have geometric surface area between 5,000 m²/m³ to 15,000 m²/m³.

The foam substrate can be any shape, such as particles, rods, sheets screens, tube, tube bundles, plates, rings, perforated sheets, honeycombs, and mixtures thereof. In certain embodiments, the substrate supported catalyst is irregular in shape. In certain embodiments, the foam substrate is in the shape of a sheet. The sheet can be between 0.1 mm to 50 mm, 0.1 mm to 40 mm, 0.1 mm to 30 mm, 0.1 mm to 20 mm, 0.1 mm to 10 mm, 0.1 mm to 8 mm, 0.1 mm to 7 mm, 1 mm to 7 mm, 1 mm to 5 mm, 0.1 mm to 5 mm, or 0.1 mm to 5 mm in thickness and the width and height of the sheet can be adjusted as required for the specific application.

The TiO₂ photocatalyst layer can have a thickness ranging of from 10 μm to 500 μm; 50 μm to 500 μm; 100 μm to 500 μm; 200 μm to 500 μm; or 30 μm to 500 μm.

In certain embodiments, the TiO₂ photocatalyst layer can have a mesoporous structure comprises pores that are 2-20 nm along their longest dimension.

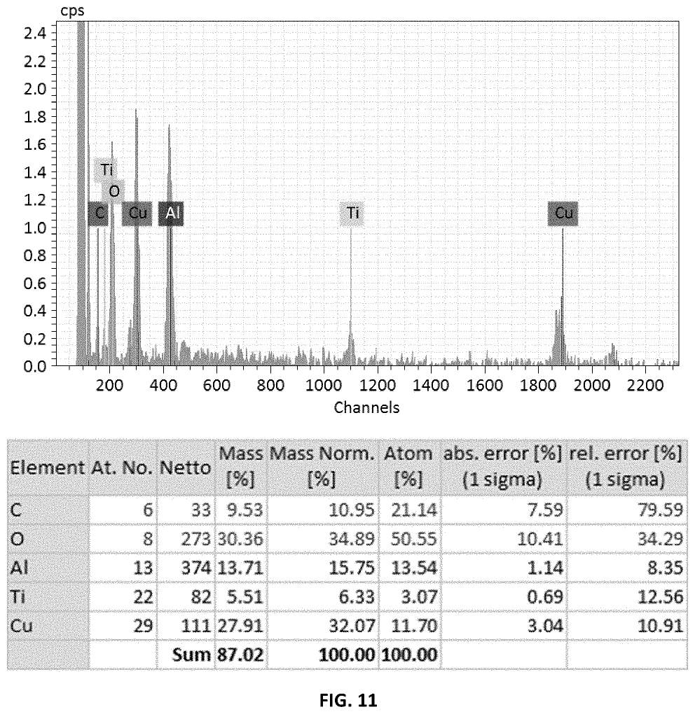

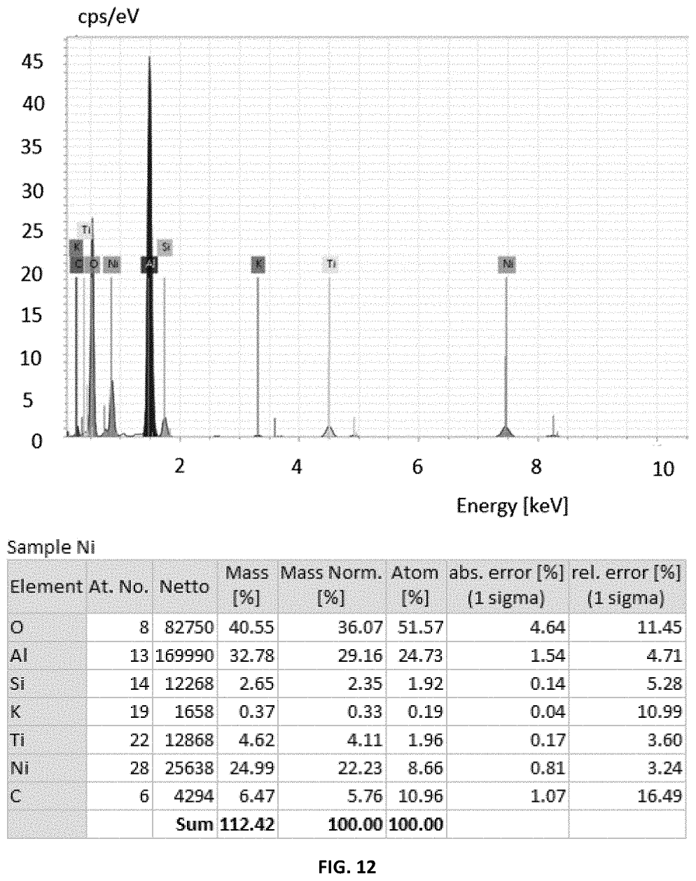

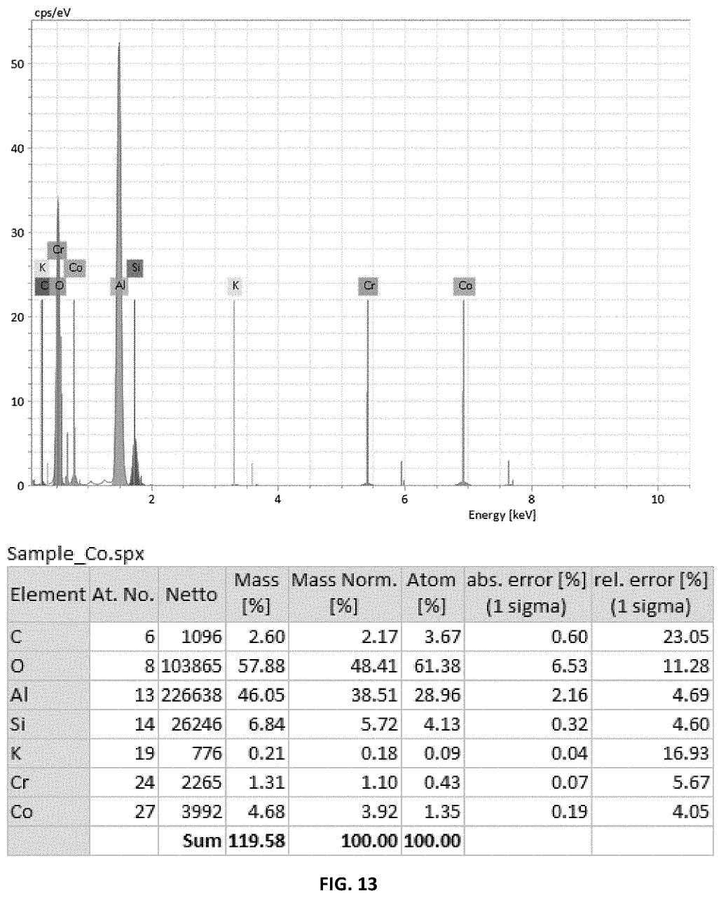

The TiO₂ photocatalyst layer can optionally be doped with a metal dopant. The concentration of the metal dopant in the TiO₂ photocatalyst layer can vary depending on stoichiometry of the reagents used to prepare the doped TiO₂ photocatalyst layer, but generally range from 0.1% to 50%, 1% to 50%, 1% to 45%, 1% to 40%, 25% to 40%, 1% to 35%, 1% to 30%, or 1% to 25%, by weight relative to the total weight of the TiO₂ and metal dopant. As shown in FIGS. 11-13, the TiO₂ photocatalyst layer comprising varying amounts of CuO, CoO, and NiO dopant can be readily prepared using the methods described herein. A person of ordinary skill in the art can facilely prepare TiO₂ photocatalyst layer doped with different concentrations of metal dopant and/or other metal dopants based on general knowledge and the methods described herein.

As shown in FIGS. 11-13, exemplary TiO₂ photocatalyst layers comprising CuO, NiO, and CoO dopants can readily be prepared using the sol-gel methods described herein having weight percentages of the metal dopant ranging from 4.6% to 37% by weight.

The metal dopant can be selected from of titanium, zirconium, copper, manganese, lanthanum, molybdenum, tungsten, vanadium, selenium, barium, cesium, tin, iron, magnesium, gold, cobalt, nickel, and palladium, or salts thereof, or mixtures thereof. In instances in which the metal dopant exists as a salt, the metal species may exists in any oxidation state, such as +1, +2, +3, or +4 oxidation state; and the salt can comprise an anion selected from halides, oxides, hydroxides, alkoxides, superoxides, nitrates, phosphates, carbides, nitrides, and mixtures thereof. In certain embodiments, the metal dopant is an oxide selected from the group consisting of titanium, zirconium, copper, manganese, lanthanum, molybdenum, tungsten, vanadium, selenium, barium, cesium, tin, iron, magnesium, gold, cobalt, nickel, and palladium. In certain embodiments, the metal dopant is CuO, CoO, NiO, or a mixture thereof.

The TiO₂ photocatalyst layer can be deposited on the foam substrate using any method known in the art. In certain embodiments, the TiO₂ photocatalyst layer is deposited on the foam substrate using a sol-gel method. In the examples below, a solution of a TiO₂ precursor and optionally the metal dopant is coated on to the foam substrate and then calcined. The TiO₂ precursor may be a titanium halide, such as titanium tetrachloride, titanylsulphate, titanyl bis(acetylacetonate), or a titanium alkoxide having the formula Ti(OR)₄, wherein R for each instance independent represents a C₁-C₆ alkyl, C₁-C₅ alkyl, C₁-C₄ alkyl, or C₂-C₄ alkyl. In certain embodiments, R is selected from the group consisting of methyl, ethyl, n-propyl, iso-propyl, n-butyl, sec-butyl, iso-butyl, tert-butyl, n-pentyl, neopentyl, isopentyl, tert-pentyl, and n-hexyl. Exemplary TiO₂ precursors include, but are not limited to titanium tetramethoxide, titanium tetraethoxide, titanium tetrapropoxide, titanium tetraisopropoxide, and titanium tetrabutoxide.

The solution of the TiO₂ precursor may further comprise a non-ionic surfactant. Exemplary non-ionic surfactants include polyoxyethylene octyl phenol (such as Triton X-100); alkylphenoxypolyethoxy (3) ethanol, polyoxyethylene (20) sorbitan monolaurate (Tween 20), polyoxyethylene (20) sorbitan monopalmitate (Tween 40), polyoxyethylene (20) sorbitan monostearate (Tween 60), polyoxyethylene (20) sorbitan tristearate (Tween 65), polyoxyethylene (20) sorbitan monooleate (Tween 80), polyoxyethylene (20) sorbitan trioleate (Tween 85), polyoxyethylene (20) palmitate (G2079), polyoxyethylene (20) lauryl ether; polyoxyethylene (23), polyoxyethylene (25) hydrogenated castor oil (G1292) and polyoxyethylene (25) oxypropylene monostearate (G2162).

In certain embodiments, the PDC reactor comprises 1, 2, 3, 4, or substrate supported catalysts. In certain embodiments, the PDC reactor comprises substrate supported catalysts disposed on the surface of 1, 2, 3, 4, or all of each of the at least two spaced air permeable plasma electrodes.

The thickness of the substrate supported catalyst can range from 1 mm to 50 mm, 1 mm to 40 mm, 1 mm to 30 mm, 1 mm to 20 mm, 1 mm to 10 mm, 1 mm to 8 mm, 1 mm to 7 mm, 1 mm to 5 mm, 3 mm to 10 mm, or 3 mm to 7 mm.

The at least two spaced air permeable plasma electrodes can be made of any electrically conductive material, such as copper, brass, or steel.

The at least two spaced air permeable plasma electrodes can be any shape that allows at least some of the air flow to pass through the electrode, such as a mesh, a grill, a perforated plate, a honeycomb, one or more rods, one or more tubes, one or more pipes, or combinations thereof. In certain embodiments, the at least two spaced air permeable plasma electrodes are mesh of conductive wires in a grid pattern, wherein the diameter of the wires can be between 0.1 to 3 mm, 0.1 to 2 mm, 0.5 to 2 mm, 0.5 to 1.5 mm, or 1 mm and the distance between the wires is 5 to 10 mm, 5 to 8 mm, 5 to 7 mm, or 6 mm. The size of each of the at least two spaced air permeable plasma electrodes will depend on the application and performance requirements of the PDC reactor, but can generally range from 1 mm² to 10,000 mm². The distance between the at least two spaced air permeable plasma electrodes can range from 1 mm to 100 mm.

An alternating current (AC) from 4 kV to 30 kV with a frequency ranging from several hundred hertz (Hz) to a few hundred kilohertz (kHz) can be applied on the electrodes to generate the dielectric barrier discharge plasma inside the plasma zone. FIG. 6 shows an exemplary wave form of the AC applied to the electrodes.

The dimensions of the plasma zone 105 can vary depending on a number of parameters, such as the desired performance, type and concentration of impurities to be removed from the air, the alternating current voltage, the thickness, etc. In certain embodiments, the length plasma zone 105, measured from the distance between the surface of each of the at least two spaced air permeable plasma electrodes, can range from 1 mm to 100 mm, 1 mm to 80 mm, 1 mm to 60 mm, 5 mm to 50 mm, 5 mm to 40 mm, 5 mm to 30 mm, 5 mm to 20 mm, or 5 mm to 15 mm.

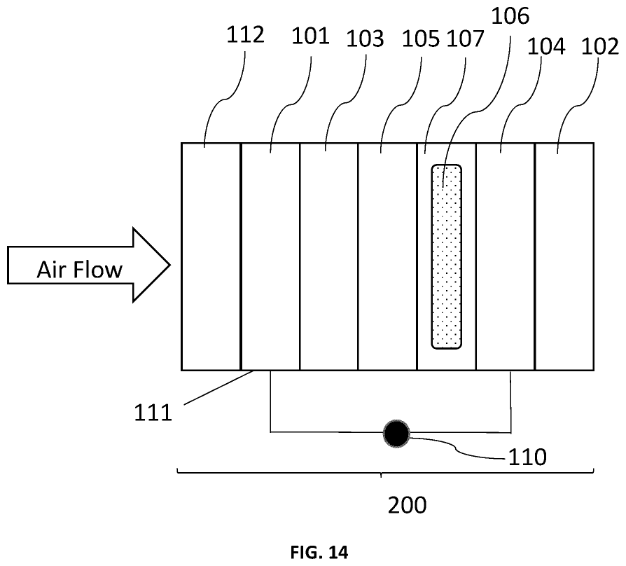

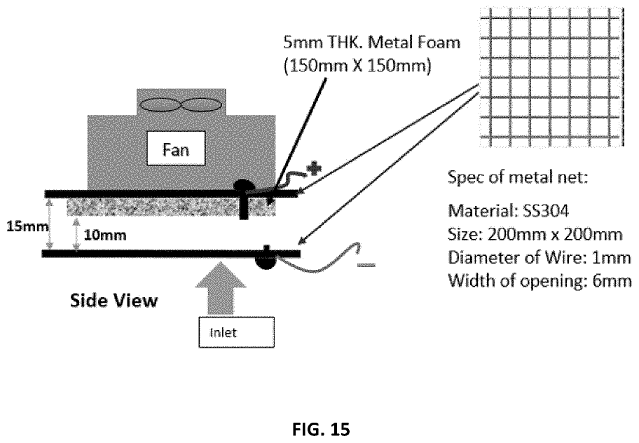

The PCR reactor described herein can be incorporated into an air purifier. Referring to FIG. 14, the air purifier may further comprise one or more fans 112, a casing 111, and a power supply 110. The one or more fans can be connected to the PCR reactor such that air flow is directed through the PCR reactor.

The present disclosure also provides methods of using the PDC reactor or air purifier described herein for treating air. Treatment of the air can comprise purifying the air and/or sanitizing the air. In certain embodiments, the method comprises directing air into the at least one air inlet, applying an alternating current voltage to the at least two spaced air permeable plasma electrodes thereby generating plasma in the plasma zone; and allowing the air to pass through the plasma zone and out of the at least one air outlet thereby forming treated air.

The term air is used broadly and encompasses any gas or gaseous mixture, e.g. nitrogen, oxygen, noble gases, and mixtures thereof. In certain embodiments, the air comprises one or more contaminants or is suspected of comprising one or more contaminants. In certain embodiments, the one or more contaminants are selected from the group consisting of a VOC, formaldehyde, CO, NO₂, H₂S, NH₃, NO, an odor, a virus, a bacteria, a mold, and a pest. The virus can be an influenza or corona virus.

Air treated using the PDC reactor or air purifier described herein can be used to improve the germination rate of seeds. In such embodiments, the method comprises: directing air into the at least one air inlet, applying an alternating current voltage to the at least two spaced air permeable plasma electrodes thereby generating plasma in the plasma zone; allowing the air to pass through the plasma zone and out of the at least one air outlet thereby forming treated air and contacting seeds with the treated air.

The seed can be any seed known in the art. In certain embodiments, the seed is any seed with poor germination, delayed germination, or substantially no germination. The seed can includes those of the Chenopodiaceae family, such as spinach (Spinacia oleracea); those of the Brassicaceae family, such as kale (Brassica oleracea); those of the Convolvulaceae family; those of the Cannaceae family; those of the Geraniaceae family; those of the Fabaceae family; those of the Myrtaceae family, such as eucalyptus(Eucalyptus sp.); those of the Malvaceae family; those of the Zingiberaceae family; those of the Primulaceae; those of the Verbenaceae family; those of the Rose family; those of the Cucurbitaceae family; those of the Asteracea family; those of the Liliaceae family; and those of the Nymphaeaceae family. In certain embodiments, the seed is a kale seed or a spinach seed.

Air treated using the PDC reactor or air purifier described herein can also be used to sanitize a surface. In such embodiments, the method comprises: directing air into the at least one air inlet, applying an alternating current voltage to the at least two spaced air permeable plasma electrodes thereby generating plasma in the plasma zone; allowing the air to pass through the plasma zone and out of the at least one air outlet thereby forming treated air and contacting a surface with the treated air, wherein the surface is contaminated or suspected of being contaminated with at least one microorganism.

The microorganism can be a unicellular or multicellular organism, such as a bacteria, a fungi, an archaea, a protist, a plant (eg, green algae), a virus, a prion, a parasite, a mold, a yeast, and an amoeba. In certain embodiments, the microorganism is a disease causing microorganism.

In certain embodiments, the microorganism is a virus. Exemplary viruses include, but are not limited to, influenza, corona virus, human immunodeficiency virus, herpes simplex virus, papilloma virus, parainfluenza virus, hepatitis, coxsackie virus, herpes zoster, measles, mumps, rubella, rabies, pneumonia, and hemorrhagic viral fever.

In certain embodiments, the microorganism is a bacteria. The bacteria can be Gram-positive bacteria or Gram-negative bacteria. In certain embodiments, the bacteria is a drug resistant bacteria. Exemplary bacteria include, Escherichia coli (E. coli), Pseudomonas aeruginosa, Udomonas aeruginosa, Staphylococcus aureus, Streptococcus pneumoniae, Mycobacterium tuberculosis, Campylobacter jejuni, Salmonella, and Shigella.

In certain embodiments, the microorganism is selected from the group consisting of E. coli, Staphylococcus aureus, H1N1, and mold.

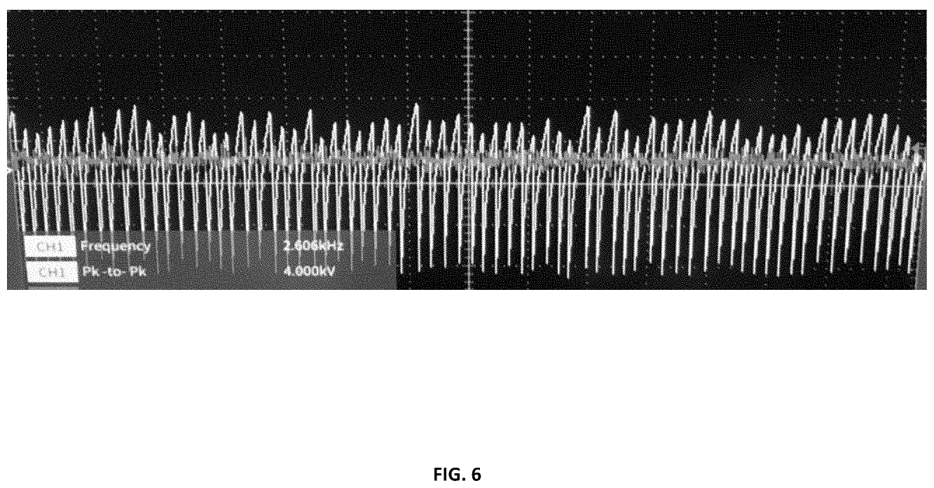

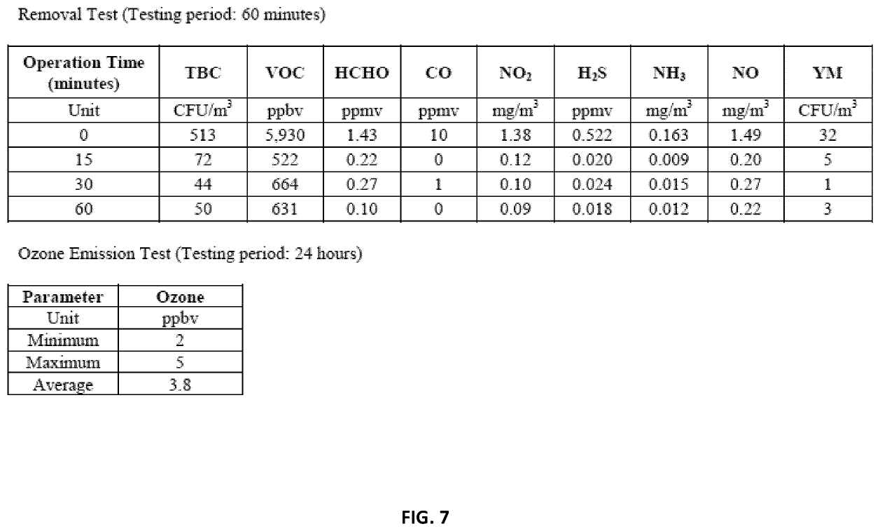

As shown by the results presented in FIG. 7, the PDC reactors described herein exhibit very high VOC, formaldehyde (HCHO), CO, NO₂, H₂S, NH₃, and NO removal performance. Over a period 60 minutes, VOCs concentration is reduced by 89%, HCHO by 93%, CO by ˜100%, NO₂ by 93%, H₂S by 97%, NH₃ by 93%, and NO by 85% with an average ozone emission concentration of 3.8 ppbv. Advantageously, a significant portion of the VOC, HCHO, CO, NO₂, H₂S, NH₃, and NO present in the gas can be removed within the first 15 minute of operation of the PDC reactors described herein.

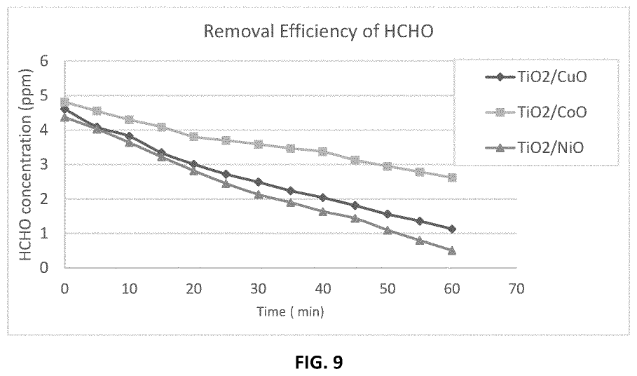

FIG. 9 shows removal efficiency of HCHO over a period of 60 minutes for PDC rectors comprising a TiO₂ photocatalyst layer doped with CuO, CoO, and NiO. Advantageously, the rate of HCHO removal for each of the tested doped TiO₂ photocatalyst layers is relatively constant with the NiO and CuO doped TiO₂ photocatalyst layer exhibiting the highest rate of HCHO removal.

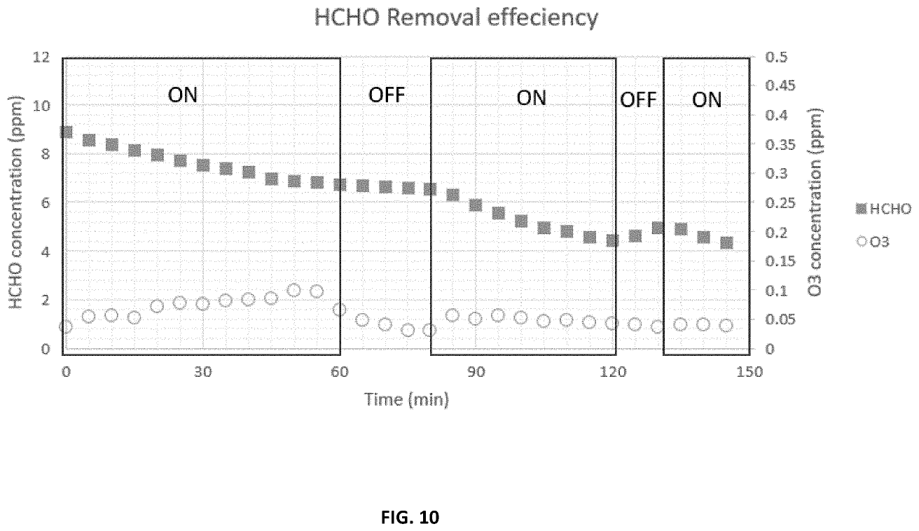

FIG. 10 shows the HCHO removal efficiency and ozone emission over a period of 150 minutes when the PDC reactor is alternated on and off.

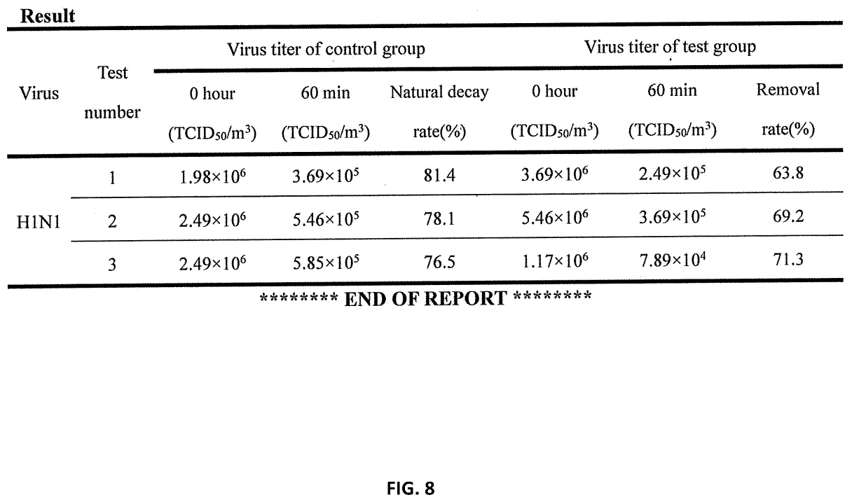

As the results in FIG. 8 demonstrate, the PDC reactors described herein can also effectively remove up to 71.3% of viruses, such as influenza (H1N1) over a period of 60 minutes of operation.

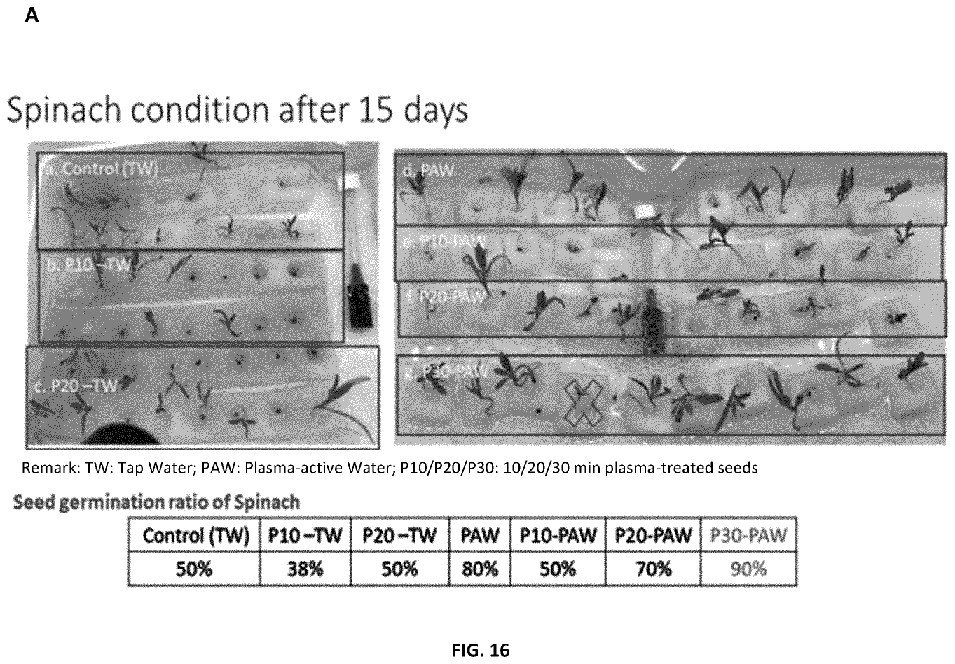

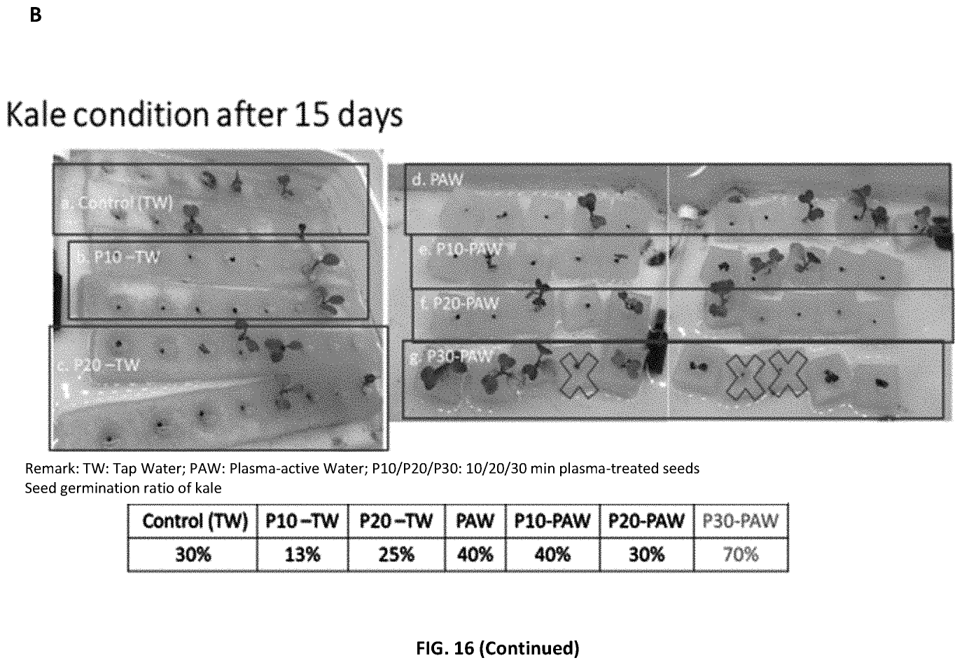

FIG. 16 demonstrates that the germination rate of spinach (A) and kale seeds (B) are enhanced by up to 40% when seeds are treated by PDC air and watered with plasma treated water (PAW).

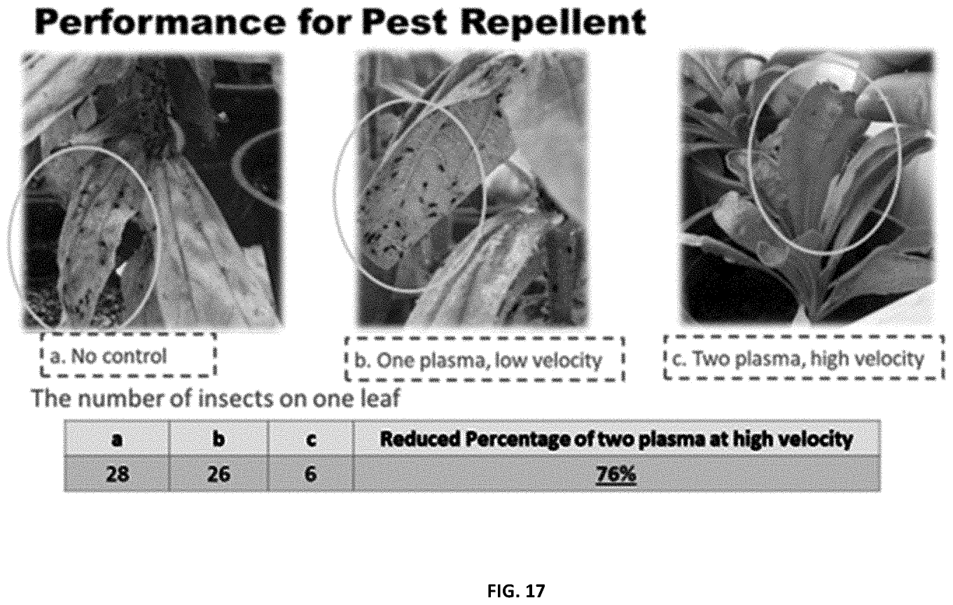

As shown by the on-site results presented in FIG. 17, the pest amount was reduced by no less than 50% under PDC reactor control at high velocity of circulation fan.

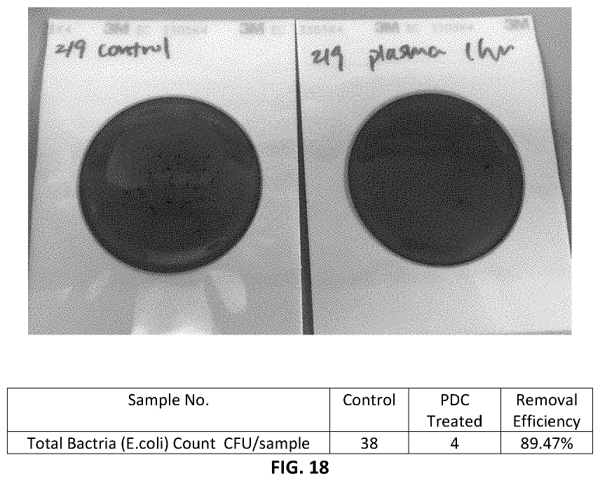

FIG. 18 shows that the 89% surface removal efficiency of bacteria can be achieved at the outlet of PDC reactor) over a period of 60 minutes of operation.

The PDC reactor described herein can be used for indoor air quality improvement in residential, commercial, and industrial air treatment environments. Such as city hall and buildings, airports and train stations, public Smoking rooms, underground malls, health care centers, clean manufacturing sites, etc.

### Examples

### 1. Preparation of TiO₂ Coating Solution

Preparation of Solution A: Triton X-100 (17.33 mL) was slowly added to cyclohexane (100 mL). Water (0.765 mL) was added dropwise to the Triton X-100 mixture and then stirred for 10 minutes. 1-hexanol (9.54 mL) was added dropwise into the Triton X-100 mixture, which turned transparent, and allowed to stir for one hour to prepare Solution A.

Preparation of Solution B: Acetylacetone (1.05 mL) was slowly added to 1-hexanol (8.0 mL). Titanium isopropoxide (5.98 mL) was added to the acetylacetone solution and stirred for 10 minutes to yield Solution B.

Preparation of Solution C: Solution A was added into Solution B was added dropwise fashion with the aid of a separating funnel and the resulting mixture was allowed to stir for 90 minutes to yield Solution C.

### 2. Preparation of Metal Doped TiO₂ Coating Solution

Preparation of Solution A: Triton X-100 (17.33 mL) was slowly added to cyclohexane (100 mL). Water (0.765 mL) was added dropwise to the Triton X-100 mixture and then stirred for 10 minutes. 1-hexanol (9.54 mL) was added dropwise into the Triton X-100 mixture, which turned transparent, and allowed to stir for one hour to prepare Solution A.

Preparation of Solution B: Acetylacetone (1.05 mL) was slowly added to 1-hexanol (8.0 mL). Titanium isopropoxide (5.98 mL) and the metal dopant (1-40% by weight relative to mass of titanium) were added to the acetylacetone sol ution and stirred for 10 minutes to yield Solution B.

Preparation of Solution C: Solution A was added into Solution B was added dropwise fashion with the aid of a separating funnel and the resulting mixture was allowed to stir for 90 minutes to yield Solution C.

### 3. Preparation of TiO₂ Coated Foam Layer by Dip Coating

The metal or ceramic foam substrate was coated with TiO₂ by dipping the substrate into Solution C at ambient temperature. The substrate was withdrawn from Solution C at a controlled speed of 5 mm/second. The coated substrate was allowed to sit for 24 hours under ambient temperature. The coated substrate was then calcined in air at 200° C. for 30 minutes and 500° C. for 2 hours with a ramp of 20° C./min. The coated substrate was then cooled to room temperature to yield the TiO₂ coated foam layer.

### 4. Preparation of TiO₂ Coated Foam Layer by Dip Spray Coating

The metal or ceramic foam substrate was spray coated with Solution C at an air pressure of 2 bar. The coated substrate was then heated in air at 150° C. for 30 minutes with a ramp of 20° C./min. The coated substrate was then calcined at 200° C. for 2 hours with a ramp of 20° C./min. The coated substrate was then cooled to room temperature to yield the TiO₂ coated foam layer.

The foregoing description of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and its practical application, thereby enabling others skilled in the art to understand the invention for various embodiments and with various modifications that are Suited to the particular use contemplated. It is intended that the scope of the invention be defined by the following claims and their equivalence.

What is claimed is:

1. A plasma driven catalyst (PDC) reactor comprising:
at least two spaced air permeable plasma electrodes for generating plasma within a plasma zone between the at least two spaced air permeable plasma electrodes by an alternating current voltage; at least one substrate supported catalyst comprising a foam substrate selected from a metal foam and a ceramic foam; and at least one TiO₂ photocatalyst layer coated on at least one surface of the foam substrate; and
at least one air inlet and at least one air outlet for allowing air to pass through the at least two spaced air permeable plasma electrodes and the plasma zone,
wherein the one substrate supported catalyst is disposed within the plasma zone and on a surface of at least one of the at least two spaced air permeable plasma electrodes.

2. The PDC reactor of claim 1, wherein the metal foam is selected from stainless steel, aluminum, nickel, copper, gold, and their alloys; and the ceramic foam is selected from silicon carbide, boron carbide, hafnium carbide, tantalum carbide, zirconia, alumina, hafnium dioxide, magnesium oxide, silicon dioxide, yttria, silicon nitride, aluminum nitride, boron nitride, hafnium nitride, titanium boride, cordierite, mullite, and mixtures thereof.

3. The PDC reactor of claim 1, wherein the foam substrate has a porosity between than 70% to 95%.

4. The PDC reactor of claim 1, wherein the foam substrate has an average pore size between 450 to 3,000 μm.

5. The PDC reactor of claim 1, wherein the foam substrate has a geometric surface area between 5,000 to 15,000 m²/m³.

6. The PDC reactor of claim 1, wherein the at least one TiO₂ photocatalyst layer further comprises a dopant selected from the group consisting of titanium, zirconium, copper, manganese, lanthanum, molybdenum, tungsten, vanadium, selenium, barium, cesium, tin, iron, magnesium, gold, cobalt, nickel, palladium, their oxides thereof, or their alloys thereof.

7. The PDC reactor of claim 1, wherein the at least one TiO₂ photocatalyst layer further comprises a dopant selected from nickel(II)oxide, copper(II)oxide, and cobalt(II)oxide.

8. The PDC reactor of claim 7, wherein the dopant is present in the at least one TiO₂ photocatalyst layer between 4% to 40% by weight.

9. The PDC reactor of claim 1, wherein the at least one TiO₂ photocatalyst layer is prepared by a sol-gel deposition method.

10. The PDC reactor of claim 1, wherein the at least two spaced air permeable plasma electrodes are mesh-plate electrodes.

11. The PDC reactor of claim 10, wherein each of the at least two spaced air permeable plasma electrodes are spaced between 1 mm to 50 mm from each other.

12. The PDC reactor of claim 1, wherein the foam substrate is a stainless steel foam having a porosity between 75% to 90%; the at least one TiO₂ photocatalyst layer further comprises 4% to 40% by weight of a dopant selected from nickel(II)oxide, copper(II)oxide, and cobalt(II)oxide; and the at least two spaced air permeable plasma electrodes are mesh-plate electrodes spaced from each other at a distance between 8 to 12 mm.

13. The PDC reactor of claim 1, wherein the foam substrate is a nickel alloy foam having a porosity between 75% to 90%; and the at least two spaced air permeable plasma electrodes are mesh-plate electrodes spaced from each other at a distance between 8 to 12 mm.

14. The PDC reactor of claim 1 further comprising an alternating current power supply connected to the at least two spaced air permeable plasma electrodes, wherein the alternating current power supply provides a frequency ranging from 0.1 to 30 kHz and the alternating voltage ranging from 1 to 10 kV.

15. The PDC reactor of claim 12 further comprising an alternating current power supply connected to the at least two spaced air permeable plasma electrodes, wherein the alternating current power supply provides a frequency ranging from 1 to 10 kHz and the alternating voltage ranging from 1 to 5 kV.

16. An air purifier comprising the PDC reactor of claim 1.

17. The air purifier of claim 16 further comprising: an electric fan positioned to direct air through the at least one air inlet.

18. A method of treating air using the PDC reactor of claim 1, the method comprising: directing air into the at least one air inlet, applying an alternating current voltage to the at least two spaced air permeable plasma electrodes thereby generating plasma in the plasma zone; and allowing the air to pass through the plasma zone and out of the at least one air outlet thereby forming treated air.

19. The method of claim 18, wherein the air comprises or is suspected of comprising one or more contaminants selected from the group consisting of a volatile organic compound, formaldehyde, CO, NO₂, H₂S, NH₃, NO, an odor, and a microorganism.

20. The method of claim 18 further comprising contacting seeds or a surface with the treated air, wherein the surface is contaminated or suspected of being contaminated with at least one microorganism.
